(19) United States
(12) Patent Application Publication
(10) Pub. No.: US 2012/0076784 A1
(43) Pub. Date: Mar. 29, 2012

(54) HIGHLY CONCENTRATED, LIQUID FORMULATIONS OF ANTI-EGFR ANTIBODIES

(76) Inventors: Susanne Matheus, Neumagen-Dhron (DE); Hanns-Christian Mahler, Wiesbaden (DE)

(21) Appl. No.: 13/311,097

(22) Filed: Dec. 5, 2011

Related U.S. Application Data
(63) Continuation of application No. 10/588,458, filed on Aug. 4, 2006, now abandoned.
(60) Provisional application No. 60/543,549, filed on Feb. 12, 2004.

(51) Int. Cl. C07K 16/28; A61K 35/02; A61K 39/395; C07K 16/30; A61P 35/04

(52) U.S. Cl. 424/133.1; 530/387.1; 530/388.15; 530/388.22; 530/387.3

(57) ABSTRACT

The invention relates to processes for the preparation of highly concentrated, liquid formulations comprising at least one anti-EGFR antibody and/or one of its variants and/or fragments, in particular monoclonal antibodies against the EGF receptor, particularly preferably of Mab C225 (cetuximab) and Mab h425 (EMD 72000), by ultrafiltration. The invention furthermore relates to highly concentrated, liquid formulations of anti-EGFR antibodies, in particular of monoclonal antibodies against the EGF receptor, particularly preferably Mab C225 (cetuximab) and Mab h425 (EMD 72000) and/or variants and/or fragments thereof, characterised in that the highly concentrated, liquid formulations have a content of anti-EGFR antibodies of 10-250, preferably 50-180 mg/ml, particularly preferably of 100-150 mg/ml, and to the use thereof.

### BACKGROUND OF THE INVENTION

The invention relates to processes for the preparation of highly concentrated, liquid formulations comprising at least one anti-EGFR antibody and/or one of its variants and/or fragments, in particular monoclonal antibodies against the EGF receptor, particularly preferably Mab C225 (cetuximab) and Mab h425 (EMD 72000), by ultrafiltration. The invention furthermore relates to highly concentrated, liquid formulations of anti-EGFR antibodies, in particular of monoclonal antibodies against the EGF receptor, particularly preferably of Mab C225 (cetuximab) and Mab h425 (EMD 72000) and/or variants and/or fragments thereof, characterised in that the highly concentrated, liquid formulations have a content of anti-EGFR antibodies of 10-250, preferably 50-180 mg/ml, particularly preferably of 100-150 mg/ml, and the to use thereof.

Advances in the area of biotechnology have made it possible in the course of the last 10 years to prepare a series of proteins for pharmaceutical application by means of recombinant DNA techniques. Protein medicaments, such as monoclonal antibodies, are used, for example, in tumour therapy, for example for specific immunotherapy or tumour vaccination. Therapeutic proteins are larger and more complex than conventional organic and inorganic active ingredients and they have complex three-dimensional structures and numerous functional groups which effect the biological activity of the protein or alternatively can cause undesired effects. During preparation, storage and transport, protein medicaments are exposed to numerous exogenous influences which can have a stability-reducing action on the protein active ingredient. It is therefore necessary to study accurately the causes and mechanisms of the specific degradation reactions in order to be able to stabilise the protein, for example through addition of certain stabilising adjuvants (see, for example, Manning M. C., Patel K., & Borchardt R. T. (1989) Stability of protein pharmaceuticals. Pharm. Res. 6, 903-918).

The literature discloses numerous formulations of therapeutic proteins. However, the requirements of the composition of a pharmaceutical preparation of protein active ingredients may be very different, and in general it is not possible, owing to specific physico-chemical properties and degradation reactions of the different proteins, to apply already established protein formulations to novel protein active ingredients. Suitable pharmaceutical formulations of these novel active ingredients are therefore still a major challenge.

Although ultrafiltration is described in the literature to date as a standard method in downstream processing in the purification of recombinant proteins (Taylor and Francis (2000) Pharmaceutical Formulation Development of Peptides and Proteins, London, p. 1-212; McPherson A. (1989) Separation Methods, Preparation and Analysis of Protein Crystals: New York, Robert E. Krieger Publishing Co., Inc., p. 1-51), advantageously high concentrations are not, however, achieved in downstream processing. In addition, dilution of the process solutions obtained can occur again due to subsequent purification and chromatography steps.

Although U.S. Pat. No. 6,252,055 describes the preparation of highly concentrated antibody formulations by means of ultrafiltration, the antibody formulations prepared in this way have, however, a high proportion of soluble aggregates of a 4%, even directly after preparation. In addition, the antibody formulations obtained are not characterised with respect to their native structure and stability, which must be regarded, for example, as very important with respect to the immunogenicity and efficacy of the antibody formulation.

The adverse effect of aggregates on increased immunogenicity and reduced efficacy as well as the reduced bioavailability of protein formulations is already known from the literature (S. A. Marshall, G. A. Lazar, A. J. Chirino, and J. R. Desjarlais. Rational design and engineering of therapeutic proteins. Drug Discovery Today 8 (5):212-221, 2003; Schellekens H. Bioequivalence and the immunogenicity of biopharmaceuticals. Nat Rev Drug Discov 1 (6):457-462, 2002).

For the above-mentioned reasons, it is clear that the preparation of liquid highly concentrated antibody formulations which are stable for a sufficiently long time is proving to be extremely difficult for the person skilled in the art. In addition, the preparation of a highly concentrated liquid formulation was unattractive to the person skilled in the art since the greatly pronounced aggregation tendency of proteins and in particular of antibodies, even in low concentration ranges, was sufficiently known (S. A. Marshall, G. A. Lazar, A. J. Chirino, and J. R. Desjarlais. Rational design and engineering of therapeutic proteins. Drug Discovery Today 8 (5):212-221, 2003). Thus, the aggregation of proteins is described in the literature as the commonest physical instability reaction (W. Wang. Instability, stabilization, and formulation of liquid protein pharmaceuticals. Int. J. Pharm. 185 (2):129-188, 1999).

Although formulations comprising Mab C225 (cetuximab) or Mab h425 (EMD 72000) are disclosed in WO03053465 and in WO03007988, the formulations disclosed in WO03053465 have, however, a relatively low protein concentration and they are not stable in the long term at room temperature. The formulations disclosed in WO03007988 likewise have a relatively low protein concentration and the preparation (Iyophilisate) has to be reconstituted before use.

The process of lyophilisation for the stabilisation of protein formulations is disclosed, for example, in WO9300807 or WO9822136, but significant disadvantages of lyophilised preparations consist in that the user has to reconstitute the lyophilisate before use, which represents a considerable source of error in the preparation before use. Since a further preparation process is added compared with liquid formulations, the process is unfavourable with respect to additional work for process development (ensuring the stability during lyophilisation), preparation (preparation costs and duration) and, for example, validation.

In the case of the formulations of low protein concentration known to date, high infusion volumes are necessary in the case of intravenous administration. The object of the invention was therefore the concentration of antibodies according to the invention, so that, through reduction of the volumes to be administered, subcutaneous administration can also be considered. Formulations to be administered subcutaneously must not exceed a volume of 1.0-1.5 ml and must furthermore be euhydric (pH 7.2 or pH 4.0-9.0) and isotonic (about 290 mOsm). A further advantage of subcutaneous formulations lies in the possibility of self-administration by the patient. However, the stability of the protein should not be impaired during the concentration, i.e. the increase in decomposition and aggregation products should be acceptable within the bounds of the specifications. Furthermore, such formulations should be free from toxicologically unacceptable substances or only comprise the latter in physiologically acceptable concentrations.

Since, due to the difficulties to be expected, already established protein formulations generally cannot be applied to new protein active ingredients, the object of the present invention was to find novel, stable, highly concentrated, liquid formulations for for therapeutic proteins, in particular monoclonal antibodies against the EGF receptor, for example Mab C225 (cetuximab) and Mab h425 (EMD 72000), which have increased stability to stress conditions, such as elevated temperature, atmospheric humidity and/or shear forces, so that their efficacy is retained during preparation, storage, transport and administration and these formulations comprise no toxicologically unacceptable adjuvants.

### SUMMARY OF THE INVENTION

Surprisingly, highly concentrated pharmaceutical anti-EGFR antibody preparations which, in a liquid formulation, facilitate protein concentrations of 10-250 mg/ml, particularly preferably of 50-180 mg/ml, particularly preferably of 100-150 mg/ml, can be obtained with the aid of ultrafiltration processes.

The formulations obtained by the ultrafiltration process are preferably stable over an extended period or they can, if necessary, be mixed with suitable stabilising adjuvants or stabilised by subsequent lyophilisation.

The formulations according to the invention are physiologically well tolerated, can be prepared easily, can be dispensed accurately and are stable throughout storage, during mechanical stress and, for example, during multiple freezing and thawing processes.

Surprisingly, it has been found that the highly concentrated anti-EGFR antibody formulations prepared by processes according to the invention comprise a monomer proportion of >99%. The resultant highly concentrated, liquid formulations according to the invention, having a concentration of 10-250 mg/ml, particularly preferably of 50-180 mg/ml, particularly preferably of 100-150 mg/ml, are physically and chemically stable, i.e. no change in the monomer content with an attendant increase in soluble aggregates occurs, which would be regarded as highly crucial with respect to the efficacy and immunogenic side effects (Schellekens H. (2002) Bio-equivalence and the immunogenicity of biopharmaceuticals.: Nat. Rev. Drug Discov. v. 1, p. 457-462). Neither do the ultrafiltration processes used cause a change in the primary structure of the protein. In addition, no disadvantages with respect to the mechanical stability and thermal stability are apparent compared with the protein formulations of low concentration. In particular, the characteristic aggregation products are also in the range of the stipulated specifications for the highly concentrated, liquid antibody formulations according to the invention.

This was unexpected since the tendency towards instability is much greater in highly concentrated protein formulations than in dilute protein formulations (Fields, G., Alonso, D., Stiger, D., Dill, K. (1992) “Theory for the aggregation of proteins and copolymers.” J. Phys. Chem. 96, 3974-3981). At a high protein concentration, the “packing density” of the protein molecules is increased. An increased number of collisions is accordingly to be assumed, and protein associations may occasionally occur. This process generally takes place due to nucleation and growth mechanisms, in which the critical nuclei are often soluble associated proteins which, however, are able to convert rapidly into insoluble protein precipitates (denatured protein) (Reithel, J. F. (1962) “The dissociation and association of protein structures”, Adv. Protein Chem. 18, 123). The size of the protein aggregates increases with increasing protein concentration, as has already been shown for β-lactoglobulin (Roefs, S. P. F. M., De Kruif, K. G. (1994) “A model for the denaturation and aggregation of β+-lactoglobulin” Eur. J. Biochem. 226, 883-889).

The anti-EGFR antibody formulations according to the invention described below are distinguished, surprisingly, by one or more advantages, selected from: high protein concentration, high stability, low aggregation tendency, low viscosity, high purity, absence of pharmaceutically unacceptable agents and thus high safety, the fact that it is well tolerated, and the possibility of direct use.

Preparation processes according to the invention described below are distinguished, surprisingly, by one or more advantages, selected from: simplicity, time and cost saving, use of pharmaceutically acceptable agents, high yield. Processes according to the invention can thus preferably be carried out significantly more simply, save time and are more cost effective than the techniques described in the literature, since, surprisingly, stable, highly concentrated, liquid anti-EGFR antibody formulations which have the above-mentioned advantages are obtained by ultrafiltration.

The invention therefore relates to processes for the preparation of highly concentrated, liquid formulations comprising at least one anti-EGFR anti-body and/or one of its variants and/or fragments by ultrafiltration. Processes according to the invention are, in particular, characterised in that the highly concentrated, liquid formulations obtained have a content of at least one anti-EGFR antibody of 10-250 mg/ml, preferably 50-180 mg/ml, particularly preferably 100-150 mg/ml.

Processes according to the invention are furthermore characterised in that the anti-EGFR antibodies are monoclonal and of murine or human origin, preferably of murine origin, and are chimeric or humanised. Particular preference is given to the anti-EGFR antibodies Mab C225 (cetuximab) or Mab h425 (EMD72000) and/or variants and/or fragments thereof.

Ultrafiltration proceses according to the invention are ultrafiltration processes such as stirred ultrafiltration and tangential flow filtration (TFF).

The ultrafiltration of the antibodies according to the invention is preferably carried out in a suitable buffer system, i.e. stabilisation of the reaction solutions, such as, for example, by detergents, is not necessary. The use of detergents in preparations for parenteral use should generally be avoided or minimised since they give rise to a not inconsiderable toxic and immunogenic potential (Sweetana S. & Akers M. J. (1996) Solubility principles and practices for parenteral drug dosage form development. PDA J. Pharm. Sci. Technol. 50, 330-342) and they can also result in a change in the secondary structure of proteins (Vermeer A. W. P. & Norde W. (2000) The influence of the binding of low molecular weight surfactants on the thermal stability and secondary structure of IgG. Colloids and Surfaces A: Physicochemical and Engineering Aspects 161, 139-150). In addition, the performance of a process for the ultrafiltration of detergent-containing formulations proves to be difficult since a disadvantageous and uncontrollable enrichment of the detergent in the product can occur owing to possible micelle formation of the detergent.

With respect to the anti-EGFR antibodies according to the invention and for the purposes of the present invention, the terms “biologically active”, “native” and “effective” are taken to mean that anti-EGFR antibodies according to the invention are able to exert their biological action even after conversion into formulations according to the invention, in particular the binding to EGFR, inhibition of the binding of ligands, in particular EGF, to the EGFR, modulation, in particular inhibition of EGFR-mediated signal transduction and prophylaxis or therapy of EGFR-mediated diseases.

anti-EGFR antibodies: anti-EGFR antibodies according to the invention are preferably monoclonal and of murine or human origin, they are particularly preferably of murine origin and are chimeric or humanised. The antibody directed against the receptor of epidermal growth factor (EGFR) is particularly preferably Mab C225 (cetuximab) or Mab h425 (EMD 72000) and/or variants or fragments thereof. Further antibodies directed against EGFR are described, for example, in EP0586002 and in J. Natl. Cancer Inst. 1993, 85: 27-33 (Mab 528).

Mab C225 (cetuximab, Erbitux™): Mab C225 (cetuximab) is a clinically proven antibody which binds to the EGF receptor. Mab C225 (cetuximab) is a chimeric antibody whose variable regions are of murine origin and whose constant regions are of human origin. It was described for the first time by Naramura et al., Cancer Immunol. Immunotherapy 1993, 37: 343-349 and in WO 96/40210 A1.

Mab h425 (EMD 72000): Mab h425 (EMD 72000) is a humanised monoclonal antibody (Mab) obtained from the murine anti-EGFR antibody 425 (Mab 425) (EP0531472). The murine monoclonal antibody Mab 425 was developed in the human carcinoma cell line A431, since it binds here to an extracellular epitope of the epidermal growth factor receptor (EGFR). It has been found that it inhibits the binding of EGF (Murthy et al., 1987). Increased expression of EGFR is found in malignant tissues from various sources, and consequently Mab 425 is a possible active ingredient for the diagnosis and therapeutic treatment of human tumours. Thus, it has been found that Mab 425 mediates tumour cytotoxicity in vitro and suppresses tumour growth of cell lines of epidermoid and colorectal carcinomas in vitro (Rodeck et al., 1987). In addition, it has been shown that Mab 425 binds to xenografts of human malignant gliomas in mice (Takahashi et al., 1987). Its humanised and chimeric forms are disclosed, for example, in EP0531472; Kettleborough et al., Protein Engineering 1991, 4: 773-783; Bier et al., Cancer Chemother Pharmacol. 2001, 47: 519-524; Bier et al., Cancer Immunol. Immunother. 1998, 46: 167-173. Mab h425 (EMD 72000) is a humanised antibody (h425) which is in clinical phase I/II and whose constant region is composed of a κ and a human γ-1 chain (EP0531472).

Human anti-EGFR antibodies can be prepared by the XenoMouse technology, as described in WO9110741, WO9402602, WO9633735. An antibody undergoing clinical trials which was prepared by this technology is, for example, also ABX-EGF (Abgenix, Crit. Rev. Oncol. Hematol. 2001, 38: 17-23; Cancer Research 1999, 59: 1236-43).

Antibody: antibody or immunoglobulin is used in the broadest sense for the purposes of the present invention and relates, in particular, to polyclonal antibodies and multispecific antibodies (for example bispecific antibodies) and particularly preferably intact monoclonal antibodies (Mab) which are biologically active, and variants and fragments thereof. The term also covers heteroantibodies which consist of two or more antibodies or fragments thereof and/or have different binding specificities and are bound to one another. Depending on the amino acid sequence of their constant regions, antibodies can be assigned to different “antibody (immunoglobulin) classes: IgA, IgD, IgE, IgG and IgM. A number of these can be further subdivided into sub-classes (isotypes), for example IgG1, IgG2, IgG3, IgG4, IgA1 and IgA2. Antibodies usually have a molecular weight of about 150 kDa, consist of two identical light chains (L) and two identical heavy chains (H). Monoclonal antibodies are obtained from a population of homogeneous cells. They are highly specific and directed against a single epitope, while polyclonal antibodies cover different antibodies which are directed against different epitopes. Processes for the preparation of monoclonal antibodies include, for example, the hybridoma method described by Kohler and Milstein (Nature 256, 495 (1975)) and in Burdon et al., (1985) “Monoclonal Antibody Technology, The Production and Characterization of Rodent and Human Hybridomas”, Eds, Laboratory Techniques in Biochemistry and Molecular Biology, Volume 13, Elsevier Science Publishers, Amsterdam. They can be prepared, in particular, by known recombinant DNA techniques (see, for example, U.S. Pat. No. 4,816,567). Monoclonal antibodies can also be isolated from phage antibody libraries, for example with the aid of the techniques described in Clackson et al. (Nature, 352: 624-628 (1991)) and Marks et al. (J. Mol. Biol., 222:58, 1-597(1991)).

Variants and fragments: variants (muteins) of antibodies are structurally related proteins, for example those which can be obtained by modification of the primary sequence (amino acid sequence), by glycoengineering (variants of the glycosylation sites or structures, also deglycosylated proteins), by PEGylation, by preparation in modified host cells or by other techniques. Variants according to the invention are not restricted here to the above examples, but instead include all variants of antibodies according to the invention which are known to the person skilled in the art. Fragments (partial segments) of antibodies are cleavage products of antibodies obtained, for example, by limited enzymatic digestion with the aid of papain, pepsin and plasmin or by preparation of the partial segments by genetic engineering. Typical partial segments are, for example, the bivalent F(ab′)₂ fragment, the monovalent Fab fragment and the Fc fragment. (Lottspeich F., H. Zorbas (ed.). Bioanalytik [Bioanalysis], Heidelberg; Berlin: Spektrum Akademischer Verlag GmbH, (1998) pp. 1035). Fragments according to the invention are not restricted here to the above examples, but instead include all fragments of antibodies according to the invention which are known to the person skilled in the art.

Pharmaceutical preparation: the terms pharmaceutical formulation and pharmaceutical preparation are used synonymously for the purposes of the present invention.

As used here, “pharmaceutically tolerated” relates to medicaments, excipients, adjuvants, stabilisers, solvents and other agents which facilitate the administration of the pharmaceutical preparations obtained therefrom to a mammal without undesired physiological side effects, such as nausea, dizziness, digestion problems or the like.

In pharmaceutical preparations for parenteral administration, there is a requirement for isotonicity, euhydria and tolerability and safety of the formulation (low toxicity), of the adjuvants employed and of the primary packing. Surprisingly, highly concentrated, liquid anti-EGFR antibody formulations according to the invention preferably have the advantage that direct use is possible, since physiologically acceptable agents are used for the preparation. The preparation of highly concentrated, liquid anti-EGFR antibody formulations according to the invention with preferably simultaneously a high yield of native and pharmaceutically acceptable protein of high purity is thus preferably simple, time-saving and inexpensive.

Ultrafiltration is a pressure-driven semipermeable membrane process for the separation of dissolved and suspended materials. The separation principle is based on the size and dimensions of the molecule, i.e. substances which are smaller than the pore size enter the filtrate (permeate), while substances which are larger than the pore size remain in the retentate (concentrate). The force needed to carry out the separation can be applied, for example, by centrifugal forces, a gas pressure source (for example nitrogen) or a membrane pump.

Highly concentrated, liquid anti-EGFR antibody formulations according to the invention can preferably be prepared by concentrating an anti-EGFR antibody-containing solution according to the invention by means of an ultrafiltration process. To this end, a solution having a defined concentration of anti-EGFR antibodies according to the invention (for example for C225: 0.01 to 150 mg/ml, preferably 2 to 100 mg/ml, particularly preferably about 20 mg/ml, for EMD 72000: 0.01 to 150 mg/ml, preferably 5 to 100 mg/ml, particularly preferably about 20 mg/ml), as is obtained in the preparation thereof, is advantageously introduced into the ultrafiltration unit and subjected to a concentration process under defined, controlled pressure conditions. If the antibody is in the form of a solid, for example a lyophilisate, the highly concentrated, liquid formulation according to the invention can be prepared by firstly dissolving anti-EGFR antibodies according to the invention in water or an aqueous solution comprising one or more of the other ingredients and subsequently subjecting the solution to the ultrafiltration process.

The product obtained by the ultrafiltration process can subsequently be stabilised by addition of the adjuvants listed below. The resultant solution comprising the respective antibody is adjusted to a pH of 4 to 10, preferably pH 5 to 9, sterile-filtered and, if necessary, possibly converted into a solid form by a subsequent lyophilisation step for stabilisation. The sequence of addition of the various adjuvants or the antibody according to the invention is substantially independent of the preparation process and is at the discretion of the person skilled in the art.

The anti-EGFR antibodies are preferably present in biologically active form in highly concentrated, liquid formulations according to the invention, and denaturing of the antibodies preferably does not occur during processes according to the invention. Thus, the biological efficacy of the protein is preferably retained.

Polyether sulfone (PES) or regenerated cellulose, for example, can be used as ultrafiltration membranes in processes according to the invention: the theoretically conceivable cut-off is in the range between 5 and 500 kDa, preferably between 10 and 100 kDa, particularly preferably between 30 and 50 kDa.

The centrifugal forces used for Ultrafree centrifuge tubes (Millipore) are in the range from 1-20,000*g, preferably in the range from 1000-12,000*g, particularly preferably 2000*g. The gas pressure used in the Amicon stirred cell (Millipore) is in the range from 0.1-5 psi, preferably 4 psi. The entry pressure used in the Labscale TFF system (Millipore) is in the range from 0.1-85 psi, preferably in the range from 10-30 psi, particularly preferably 20 psi. The exit pressure used in the Labscale TFF system (Millipore) is in the range from 0.1-85 psi, preferably in the range from 5-20 psi, particularly preferably 10 psi.

The following buffers, for example, can be used in processes according to the invention: phosphate buffers: Na (or K) phosphate; possible pH about 6.0-8.2; citrate buffers: Na citrate or citric acid, possible pH about 2.2-6.5, succinate buffers pH about 4.8-6.3, acetate buffers, for example sodium acetate, pH about 2.5-6.0; histidine buffers pH about 6.0-7.8; glutamic acid pH 8.0 to 10.2; glycine (N,N-bis(2-hydroxyethyl)glycine) pH about 8.6 to 10.6; glycinate buffers pH about 6.5 -7.5; imidazole pH 6.2 to 7.8; potassium chloride pH about 1.0 to 2.2; lactate buffers pH about 3.0-6.0; maleate buffers pH about 2.5-5.0; tartrate buffers pH about 3.0-5.0; Tris: pH about 6.8-7.7; phosphate-citrate buffers. The addition of isotonic agents for effecting isotonicity is also conceivable (for example NaCl (or KCl) or also other salts).

Above-mentioned buffers can be used, for example, in the following concentrations in processes according to the invention: 1 mM to 200 mM, preferably 2-20 mM, particularly preferably about 10 mM.

The following pH ranges can preferably be used:

pH 4-10, preference is given to pH=IEP +/−2 pH units (2 pH units around the isoelectric point of the protein).

The following isotonic agents can preferably be used (usual concentrations): sodium chloride about 5 mM-305 mM; potassium chloride; glucose; glycerol; dextrose 4-5.5 mM; sodium sulfate 1-1.6 mM.

The following substances can preferably be used for reducing the viscosity: sodium chloride, arginine hydrochloride, sodium thiocyanate, ammonium thiocyanate, ammonium sulfate, ammonium chloride, calcium chlorides, zinc chlorides, sodium acetate.

The following stabilisers can preferably be used:

1) Amino Acids

(About 1-100 mg/ml, particularly preferably 3-10 mg/ml, as hydrochloride) arginine, ornithine, lysine, histidine, glutamic acid, aspartic acid, isoleucine, leucine, alanine, phenylalanine, tyrosine, tryptophan, methionine, serine, proline.

2) Sugars and Sugar Alcohols

(About 1-200 mg/ml, particularly preferably 30-65 mg/ml) sucrose, lactose, glucose, mannose, maltose, galactose, fructose, sorbose, raffinose, trehalose, glucosamine, N-methylglucosamine, galactosamine, neuramic acid.

3) Antioxidants

Acetone sodium bisulfite 0.2%, ascorbic acid 0.01%, ascorbic acid ester 0.015%, butylhydroxyanisole (BHA) 0.02%, butylhydroxytoluene (BHT) 0.02%, cysteine 0.5%, nordihydroguaiaretic acid (NDGA) 0.01%, monothioglycerol 0.5%, sodium bisulfite 0.15%, sodium metabisulfite 0.2%, tocopherols 0.5%, glutathione 0.1%.

4) Preservatives

m-Cresol about 0.1-0.3%, chlorocresol about 0.1-0.3%, phenol about 0.5%, benzyl alcohol about 1.0-2.0%, methylparaben about 0.2%, propylparaben about 0.02%, butylparaben about 0.015%, chlorobutanol about 0.25-0.5%, phenylmercury nitrate about 0.002%, phenylmercury acetate about 0.002%, thimersal about 0.01-0.02%, benzalkonium chloride about 0.01%, benzethonium chloride about 0.01%.

5) Cyclodextrins

For example hydroxypropyl-β-cyclodextrin, sulfobutylethyl-β-cyclodextrin, γ-cyclodextrin.

6) Albumins

Human serum albumin (HSA), bovine serum albumin (BSA):

7) Polyhydric Alcohols

Glycerol, ethanol, mannitol.

8) Salts

Acetate salts (for example sodium acetate), magnesium chloride, calcium chloride, tromethamine, EDTA (for example Na EDTA).

The invention also encompasses all hydrates, salts and derivatives of the above-mentioned agents that are known and conceivable to the person skilled in the art.

The invention furthermore relates to highly concentrated, liquid formulations comprising at least one anti-EGFR antibody and/or one of its variants and/or fragments. These highly concentrated, liquid anti-EGFR antibody formulations can be prepared by ultrafiltration processes described above. Further conceivable concentration processes are chromatographic processes, such as, for example, size exclusion chromatography (for example gel filtration), affinity chromatography (for example protein A chromatography) or ion exchange chromatography, membrane separation processes, such as, for example, dialysis, electrodialysis, microfiltration, reverse osmosis, electrophoretic processes or drying processes, such as, for example, nitrogen gas drying, vacuum oven drying, lyophilisation, washing in organic solvents and subsequent air drying, liquid-bed drying, fluidised-bed drying, spray drying, roll drying, layer drying, air drying at room temperature and subsequent reconstitution in a smaller volume of solvent.

Highly concentrated, liquid anti-EGFR antibody formulations according to the invention are, in particular, characterised in that they have a content of at least one anti-EGFR antibody of 10-250 mg/ml, preferably of 50-180 mg/ml, particularly preferably of 100-150 mg/ml.

Highly concentrated, liquid formulations according to the invention are, in particular, characterised in that the anti-EGFR antibodies are monoclonal and of murine or human origin, preferably of murine origin, and are chimeric or humanised. The anti-EGFR antibodies are particularly preferably Mab C225 (cetuximab) or Mab h425 (EMD72000) and/or variants and/or fragments thereof.

The invention furthermore relates to highly concentrated, liquid formulations comprising at least one anti-EGFR antibody and/or one of its variants and/or fragments obtainable by processes according to the invention, i.e. by ultrafiltration processes described above.

The invention additionally relates to highly concentrated, liquid anti-EGFR antibody formulations according to the invention as storage-stable medicaments.

Highly concentrated, liquid anti-EGFR antibody formulations according to the invention may, in addition to antibodies according to the invention, optionally comprise excipients and/or adjuvants and/or further pharmaceutical active ingredients.

Processes according to the invention preferably enable highly concentrated formulations to be prepared without unfavourable, undesired aggregation of the antibodies according to the invention occurring. Thus, ready-to-administer solutions having a high active ingredient content can be prepared with the aid of processes according to the invention according to the invention. Very highly concentrated formulations of protein active ingredients have recently increasingly been required. Most antibodies employed for therapy are administered in a dose in the mg/kg region. A high dose and small volumes to be administered (for example about 1 to 1.5 ml in the case of subcutaneous administration) show the need for highly concentrated protein preparations having concentrations of greater than 100 mg/ml. In addition, highly concentrated protein formulations may have considerable advantages in preclinical tests for investigation of the acceptability and efficacy in vitro and in vivo (on an animal model), in clinical tests for investigation of the acceptability and efficacy in humans and in clinical use of the product (in particular in the case of subcutneous administration). Their advantages consist, in particular, in that a smaller volume of the preparation has to be used. In contrast to infusion or injection of protein medicaments of relatively low concentration, subcutaneous administration of, for example, protein medicaments is thus possible for the patient. Subcutaneous administration of protein medicaments can have various reasons. For example, specific targeting may be desired in connection with a “therapeutic window”. Furthermore, subcutaneous administration has the advantage that the patient can carry out the administration himself without having to rely on medical personnel. The example of insulin clearly exhibits these advantages. However, since the injections for subcutaneous administration can be a maximum of 1-1.5 ml, highly concentrated protein formulations comprising more than 100 mg/ml are frequently necessary.

Surprisingly, highly concentrated, liquid anti-EGFR antibody formulations which do not have the above-mentioned disadvantages at protein concentrations of 10-250 mg/ml, preferably of 50-180 mg/ml, particularly preferably of 100-150 mg/ml, can be obtained with the aid of processes according to the invention.

The limit in the case of known highly concentrated immunoglobulin formulations is normally 2-50 mg/ml in the case of ready-to-use liquid antibody formulations (Humira®)

Using the processes according to the invention, however, significantly more highly concentrated and nevertheless stable formulations can also be prepared, which was unexpected. Thus, processes according to the invention enable highly concentrated stable antibody formulations to be obtained which have a reduced viscosity and aggregation tendency compared with known highly concentrated, liquid antibody formulations and thereby thereby the handling in the case of parenteral administration is simplified.

The formulations according to the invention can advantageously be used to prepare antibody-containing solutions having a pH of 4 to 10, preferably having a pH of 5 to 9, and an osmolality of 250 to 350 mOsmol/kg. Formulations according to the invention can thus be directly administered intravenously, intraarterially and also subcutaneously substantially without pain. In addition, the preparation can also be added to infusion solutions, such as, for example, glucose solution, isotonic saline solution or Ringer's solution, which may also comprise further active ingredients, so that relatively large amounts of active ingredient can also be administered.

The formulations according to the invention are physiologically well tolerated, can be prepared easily, can be dispensed accurately and are preferably stable with respect to content, decomposition products and aggregates throughout storage and transport and during multiple freezing and thawing processes. They can preferably be stored in a stable manner over an extended period at refrigerator temperature (2-8° C.) and at room temperature (23-27° C.) and 60% relative atmospheric humidity (RH). Formulations according to the invention are also preferably comparatively stable at elevated temperatures and atmospheric humidities.

The term “effective amount” denotes the amount of a medicament or of a pharmaceutical active ingredient which causes a biological or medical response in a tissue, system, animal or human which is sought or desired, for example, by a researcher or physician.

In addition, the term “therapeutically effective amount” denotes an amount which, compared with a corresponding subject who has not received this amount, has the following consequence: improved treatment, healing, prevention or elimination of a disease, syndrome, disease state, condition, disorder or prevention of side effects or also the reduction in the progress of a disease, condition or disorder. The term “therapeutically effective amount” also encompasses the amounts which are effective for increasing normal physiological function.

Medicaments can be administered in the form of dosage units which comprise a predetermined amount of active ingredient per dosage unit. A unit of this type can comprise, for example, 0.5 mg to 1 g, preferably 1 mg to 800 mg, of an active ingredient according to the invention, depending on the disease state treated, the method of administration and the age, weight and health of the patient. Preferred dosage unit formulations are those which comprise a daily dose or part-dose, as indicated above, or a corresponding fraction thereof of an active ingredient. Furthermore, medicaments of this type can be prepared by means of one of the processes generally known in the pharmaceutical sector.

Medicaments can be adapted for administration by any desired suitable route, for example by the oral (including buccal or sublingual), rectal, pulmonary, nasal, topical (including buccal, sublingual or transdermal), vaginal or parenteral (including subcutaneous, intramuscular, intravenous or intradermal) routes. Medicaments of this type can be prepared by means of all processes known in the pharmaceutical sector by, for example, combining the active ingredient with the excipient(s) or adjuvant(s).

Parenteral administration is preferably suitable for administration of the medicaments according to the invention. In the case of parenteral administration, intravenous, subcutaneous or intradermal administration are particularly preferred. In the case of intravenous administration, the injection can take place directly or also as an addition to infusion solutions.

Medicaments according to the invention for subcutaneous or intradermal administration are particularly suitable since the small volumes to be administered that are necessary for subcutaneous administration can be achieved with the aid of the highly concentrated, liquid formulations according to the invention.

Subcutaneous administration has the advantage that the patient can administer the medicament himself without expert medical aid. Anti-EGFR antibody formulations according to the invention are also suitable for the preparation of medicaments to be administered parenterally having slow, sustained and/or controlled release of active ingredient, for example also for the preparation of delayed-release formulations, which are advantageous for the patient since administration is only necessary at relatively long time intervals. Pharmaceutical preparations according to the invention can also be injected directly into the tumour and thus develop their action directly at the site of action as intended.

The medicaments adapted for parenteral administration include aqueous and non-aqueous sterile injection solutions comprising antioxidants, buffers, bacteriostatics and solutes, by means of which the formulation is rendered isotonic with the blood of the recipient to be treated; as well as aqueous and non-aqueous sterile suspensions, which can comprise suspension media and thickeners. The formulations can be delivered in single-dose or multidose containers, for example sealed ampoules and vials, and stored in the freeze-dried (lyophilised) state, so that only the addition of the sterile carrier liquid, for example water for injection purposes, immediately before use is necessary. Injection solutions and suspensions prepared in accordance with the recipe can be prepared from sterile powders, granules and tablets.

The anti-EGFR antibody formulations according to the invention can also be administered in the form of liposome delivery systems, such as, for example, small unilamellar vesicles, large unilamellar vesicles and multi-lamellar vesicles. Liposomes can be formed from various phospholipids, such as, for example, cholesterol, stearylamine or phosphatidylcholines.

Medicaments adapted for topical administration can be introduced into the formulations according to the invention formulated as ointments, creams, suspensions, lotions, solutions, pastes, gels, sprays, aerosols or oils.

For treatment of the eye or other external tissue, for example mouth and skin, the formulations are preferably introduced into topical ointment or cream and applied. In the case of formulation to give an ointment, formulations according to the invention can either be introduced into a paraffinic or a water-miscible cream base. Alternatively, a formulation according to the invention can be formulated to give a cream with an oil-in-water cream base or a water-in-oil base.

The medicaments adapted for topical administration to the eye include eye drops.

Medicaments adapted for rectal administration can be delivered in the form of suppositories or enemas.

Medicaments adapted for administration by inhalation encompass finely particulate dusts or mists which can be produced by means of various types of pressurised dispensers with aerosols, atomisers or insufflators.

Medicaments adapted for vaginal administration can be delivered as pessaries, tampons, creams, gels, pastes, foams or spray formulations.

It goes without saying that, besides the constituents particularly mentioned above, the medicaments according to the invention may also comprise other agents usual in the sector with relation to the particular type of pharmaceutical formulation.

The invention furthermore relates to sets (kits) consisting of separate packs of
a) a formulation according to the invention comprising an effective amount of an anti-EGFR antibody, preferably of a monoclonal anti-EGFR anti-body, particularly preferably of Mab C225 (cetuximab) or Mab h425 (EMD 72000) and/or variants or fragments thereof, and
b) a formulation comprising an effective amount of a further medicament active ingredient.

The set comprises suitable containers, such as boxes or cartons, individual bottles, bags or ampoules. The set may, for example, comprise separate ampoules each containing a formulation according to the invention comprising an effective amount of an anti-EGFR antibody according to the invention and a formulation of a further medicament active ingredient in dissolved or lyophilised form.

A therapeutically effective amount of an anti-EFGR antibody according to the invention depends on a number of factors, including, for example, the age and weight of the patient, the precise disease state requiring treatment, and its severity, the nature of the formulation and the method of administration, and is ultimately determined by the treating doctor or veterinarian. However, an effective amount of an anti-EFGR antibody according to the invention for the treatment of neoplastic growth, for example intestinal or breast carcinoma, is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg would usually be between 70 and 700 mg, where this amount can be given as a single dose per day or usually in a series of part-doses (such as, for example, two, three, four, five or six) per day, so that the total daily dose is the same. The suitable antibody titre is determined by methods known to the person skilled in the art. The dose proposed for administration is generally sufficient to achieve the desired tumour-inhibiting action. However, the dose should also be chosen to be as low as possible so that no side effects, such as undesired cross-reactions, anaphylactic reactions or the like, occur.

Medicaments according to the invention can be used, in particular, for the prophylaxis and/or for the treatment of diseases and disease states.

The invention therefore furthermore also relates to the use of highly concentrated, liquid anti-EGFR antibody formulations according to the invention for the preparation of a medicament for the treatment and/or prophylaxis of tumours and/or tumour metastases, where the tumour is selected from the group consisting of brain tumour, tumour of the urogenital tract, tumour of the lymphatic system, stomach tumour, laryngeal tumour, monocytic leukaemia, lung adenocarcinoma, small-cell lung carcinoma, pancreatic cancer, glioblastoma and breast carcinoma.

It has been shown in various in-vitro and in-vivo studies that blockage of the EGFR by antibodies against tumours at various levels, for example by inhibiting the proliferation of cancer cells, reducing tumour-mediated angiogenesis, inducing cancer cell apoptosis and increasing the toxic effects of radiation therapy and conventional chemotherapy.

Medicaments comprising formulations according to the invention are able effectively to regulate, modulate or inhibit EGFR and can therefore be employed for the prevention and/or treatment of diseases in connection with unregulated or disturbed EGFR activity. In particular, the anti-EGFR antibody formulations according to the invention can therefore be employed in the treatment of certain forms of cancer and in diseases caused by pathological angiogenesis, such as diabetic retinopathy or inflammation.

The invention therefore furthermore relates to the use of formulations according to the invention for the preparation of a medicament for the treatment and/or prophylaxis of diseases caused, mediated and/or propagated by EGFR and/or by EGFR-mediated signal transduction.

Medicaments according to the invention are particularly suitable for the treatment and/or prophylaxis of cancer, including solid carcinomas, such as, for example, carcinomas (for example of the lungs, pancreas, thyroid, bladder or colon), myeloid diseases (for example myeloid leukaemia) or adenomas (for example villous colonic adenoma), pathological angiogenesis and metastatic cell migration. The medicaments are furthermore useful in the treatment of complement activation-dependent chronic inflammation (Niculescu et al. (2002) Immunol. Res., 24:191-199) and immunodeficiency induced by HIV-1 (human immunodeficiency virus type 1) (Popik et al. (1998) J Virol, 72: 6406-6413).

In addition, the present medicaments are suitable as pharmaceutical active ingredients for mammals, in particular for humans, in the treatment of EGFR-induced diseases. The term “EGFR-induced diseases” relates to pathological states which are dependent on EGFR activity. EGFR is involved either directly or indirectly in the signal transduction pathways of various cell activities, including proliferation, adhesion and migration, as well as differentiation. The diseases associated with EGFR activity include the proliferation of tumour cells, pathological neovascularisation, which promotes the growth of solid tumours, neovascularisation in the eye (diabetic retinopathy, age-induced macular degeneration and the like) and inflammation (psoriasis, rheumatoid arthritis and the like).

The diseases discussed here are usually divided into two groups, hyperproliferative and non-hyperproliferative diseases. In this connection, psoriasis, arthritis, inflammation, endometriosis, scarring, benign prostate hyperplasia, immunological diseases, autoimmune diseases and immunodeficiency diseases are regarded as non-cancerous diseases, of which arthritis, inflammation, immunological diseases, autoimmune diseases and immunodeficiency diseases are usually regarded as non-hyperproliferative diseases.

In this connection, brain cancer, lung cancer, squamous cell carcinoma, bladder cancer, stomach cancer, pancreatic cancer, liver cancer, kidney cancer, colorectal cancer, breast cancer, head cancer, neck cancer, oesophageal cancer, gynaecological cancer, thyroid cancer, lymphomas, chronic leukaemia and acute leukaemia are to be regarded as cancerous diseases, all of which are usually counted amongst the group of hyperproliferative diseases. In particular, cancerous cell growth and in particular cancerous cell growth mediated directly or indirectly by EGFR is an disease which represents a target of the present invention.

It can be shown that the medicaments according to the invention have an in-vivo antiproliferatve action in a xenotransplant tumour model. The medicaments according to the invention are administered to a patient with a hyperproliferative disease, for example for inhibiting tumour growth, for reducing the inflammation associated with a lymphoproliferative disease, for inhibiting transplant rejection or neurological damage owing to tissue repair, etc. The present medicaments are useful for prophylactic or therapeutic purposes. As used herein, the term “treat” is used as reference both to the prevention of diseases and the treatment of existing conditions. The prevention of proliferation is achieved by administration of the medicaments according to the invention before development of the evident disease, for example for preventing tumour growth, preventing metastatic growth, reducing restenosis associated with cardiovascular surgery, etc. Alternatively, the medicaments are used for the treatment of chronic diseases by stabilising or improving the clinical symptoms of the patient.

The host or patient can belong to any mammalian species, for example a primate species, particularly humans; rodents, including mice, rats and hamsters; rabbits; horses, cows, dogs, cats, etc. Animal models are of interest for experimental studies, providing a model for the treatment of human disease.

The receptivity of a certain cell to treatment with the medicaments according to the invention can be determined by in-vitro tests. Typically, a culture of the cell is incubated with a medicament according to the invention at different concentrations for a period which is sufficient to enable the active ingredients to induce cell death or inhibit migration, usually between about one hour and one week. In-vitro tests can be carried out using cultivated cells from a biopsy sample. The viable cells remaining after the treatment are then counted.

The dose varies depending on the specific medicaments used, the specific disease, the patient status, etc. Typically, a therapeutic dose is sufficient in order considerably to reduce the undesired cell population in the target tissue, while the viability of the patient is maintained. The treatment is generally continued until a considerable reduction has occurred, for example a reduction of at least about 50% of the specific cell count, and can be continued until essentially no undesired cells are detected in the body.

Various assay systems are available for identification of EGFR inhibitors. In the scintillation proximity assay (Sorg et al., J. of. Biomolecular Screening, 2002, 7, 11-19) and the flashplate assay, the radioactive phosphorylation of a protein or peptide as substrate is measured using γATP. In the presence of an inhibitory compound, a reduced radioactive signal or none at all can be detected. Furthermore, homogeneous time-resolved fluorescence resonance energy transfer (HTR-FRET) and fluorescence polarisation (FP) technologies are useful as assay methods (Sills et al., J. of Biomolecular Screening, 2002, 191-214).

Other non-radioactive ELISA assay methods use specific phospho-anti-bodies (phospho-ABs). The phospho-AB only binds the phosphorylated substrate. This binding can be detected using a second peroxidase-conjugated anti-sheep antibody by chemiluminescence (Ross et al., 2002, Biochem. J., just about to be published, manuscript BJ20020786).

There are many diseases and disease states associated with deregulation of cell proliferation and of cell death (apoptosis). The diseases and disease states which can be treated, prevented or ameliorated by medicaments according to the invention include the diseases and disease states listed below, but are not restricted thereto. The medicaments according to the invention are useful in the treatment and/or prophylaxis of a number of different diseases and disease states which involve proliferation and/or migration of smooth muscle cells and/or inflammation cells in the intimal layer of a vessel, resulting in restricted blood flow through this vessel, for example in neointimal occlusive lesions. Occlusive transplant vessel diseases of interest include atherosclerosis, coronary vascular disease after transplantation, vein transplant stenosis, peri-anastomotic prosthesis restenosis, restenosis after angioplasty or stent placement and the like.

The present invention encompasses the use of the medicaments according to the invention for the treatment or prevention of cancer. The invention therefore particularly preferably relates to the use of liquid anti-EGFR antibody formulations according to the invention for the preparation of a medicament for the treatment and/or prophylaxis of tumours and/or tumour metastases, where the tumour is particularly preferably selected from the group consisting of brain tumour, tumour of the urogenital tract, tumour of the lymphatic system, stomach tumour, laryngeal tumour, monocytic leukaemia, lung adenocarcinoma, small-cell lung carcinoma, pancreatic cancer, glioblastoma and breast carcinoma, without being restricted thereto.

The invention furthermore relates to the use of medicaments according to the invention for the preparation of a medicament for the treatment of diseases selected from the group of cancerous diseases consisting of squamous cell carcinoma, bladder cancer, stomach cancer, liver cancer, kidney cancer, colorectal cancer, breast cancer, head cancer, neck cancer, oesophageal cancer, gynaecological cancer, thyroid cancer, lymphoma, chronic leukaemia and acute leukaemia.

The medicaments according to the invention can be administered to patients for the treatment of cancer. The present medicaments inhibit tumour angiogenesis and thus influence the growth of tumours (J. Rak et al. Cancer Research, 55:4575-4580, 1995). The angiogenesis-inhibiting properties of the medicaments according to the invention are also suitable for the treatment of certain forms of blindness associated with retinal neovascularisation.

The invention therefore also relates to the use of anti-EGFR antibody formulations according to the invention for the preparation of a medicament for the treatment and/or prophylaxis of diseases caused, mediated and/or propagated by angiogenesis.

A disease of this type involving angiogenesis is an ocular disease, such as retinal vascularisation, diabetic retinopathy, age-induced macular degeneration and the like.

The invention therefore also relates to the use of anti-EGFR antibody formulations according to the invention for the preparation of a medicament for the treatment and/or prophylaxis of diseases selected from the group consisting of retinal vascularisation, diabetic retinopathy, age-induced macular degeneration and/or inflammatory diseases.

The invention furthermore relates to the use of anti-EGFR antibody formulations according to the invention for the treatment and/or prophylaxis of diseases selected from the group consisting of psoriasis, rheumatoid arthritis, contact dermatitis, delayed hypersensitivity reaction, inflammation, endometriosis, scarring, benign prostate hyperplasia, immunological diseases, autoimmune diseases and immunodeficiency diseases.

The invention also relates to the use of anti-EGFR antibody formulations according to the invention for the treatment and/or prophylaxis of bone pathologies selected from the group consisting of osteosarcoma, osteoarthritis and rickets.

The medicaments according to the invention can furthermore be used to provide additive or synergistic effects in certain existing cancer chemotherapies and irradiations, and/or can be used to restore the efficacy of certain existing cancer chemotherapies and irradiations.

The invention therefore also relates to the use of anti-EGFR antibody formulations according to the invention for the preparation of a medicament for the treatment and/or prophylaxis of diseases in which a therapeutically effective amount of an anti-EGFR antibody according to the invention is administered in combination with a compound from the group 1) oestrogen receptor modulator, 2) androgen receptor modulator, 3) retinoid receptor modulator, 4) cytotoxic agent, 5) antiproliferative agent, 6) prenyl protein transferase inhibitors, 7) HMG-CoA reductase inhibitors, 8) HIV protease inhibitors, 9) reverse transcriptase inhibitors, 10) growth factor receptor inhibitors and 11) angiogenesis inhibitors.

The invention therefore also relates to the use of anti-EGFR antibody formulations according to the invention for the preparation of a medicament for the treatment and/or prophylaxis of diseases in which a therapeutically effective amount of an anti-EGFR antibody according to the invention is administered in combination with radiotherapy and a compound from the group 1) oestrogen receptor modulator, 2) androgen receptor modulator, 3) retinoid receptor modulator, 4) cytotoxic agent, 5) antiproliferative agent, 6) prenyl protein transferase inhibitors, 7) HMG-CoA reductase inhibitors, 8) HIV protease inhibitors, 9) reverse transcriptase inhibitors, 10) growth factor receptor inhibitors and 11) angiogenesis inhibitors.

The medicaments according to the invention can thus also be administered together with other well-known therapeutic agents that are selected for their particular utility against the condition that is being treated. Thus, for example in the case of bone conditions, combinations that would be favourable include those which comprise antiresorptive bisphosphonates, such as alendronate and risedronate, integrin blockers (as defined further below), such as αvβ3 antagonists, conjugated oestrogens used in hormone replacement therapy, such as Prempro®, Premarin® and Endometrion®; selective oestrogen receptor modulators (SERMs), such as raloxifene, droloxifene, CP-336,156 (Pfizer) and lasofoxifene, cathepsin K inhibitors and ATP proton pump inhibitors.

The present medicaments are also suitable for combination with known anti-cancer agents. These known anti-cancer agents include the following: oestrogen receptor modulators, androgen receptor modulators, retinoid receptor modulators, cytotoxic agents, antiproliferative agents, prenyl protein transferase inhibitors, HMG-CoA reductase inhibitors, HIV protease inhibitors, reverse transcriptase inhibitors, growth factor inhibitors and angiogenesis inhibitors. The present compounds are particularly suitable for administration at the same time as radiotherapy.

“Oestrogen receptor modulators” refers to compounds which interfere with or inhibit the binding of oestrogen to the receptor, regardless of mechanism. Examples of oestrogen receptor modulators include, but are not limited to, tamoxifen, raloxifene, idoxifene, LY353381, LY 117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]phenyl 2,2-dimethyl-propanoate, 4,4′-dihydroxybenzophenone-2,4-dinitrophenylhydrazone and SH646.

“Androgen receptor modulators” refers to compounds which interfere with or inhibit the binding of androgens to the receptor, regardless of mechanism. Examples of androgen receptor modulators include finasteride and other 5α-reductase inhibitors, nilutamide, flutamide, bicalutamide, liarozole and abiraterone acetate.

“Retinoid receptor modulators” refers to compounds which interfere with or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-4′-hydroxyphenyl)retinamide and N-4-carboxyphenyl retinamide.

“Cytotoxic agents” refers to compounds which result in cell death primarily through direct action on the cellular function or inhibit or interfere with cell myosis, including alkylating agents, tumour necrosis factors, intercalators, microtubulin inhibitors and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPX100, (trans,trans,trans)-bis-mu-(hexane-1,6-diamine)-mu-[diamine-platinum(II)]bis[diamine(chloro)platinum(II)] tetrachloride, diarizidinyl-spermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-de-amino-3′-morpholino-13-deoxo-10-hydroxycarminomycin, annamycin, galarubicin, elinafide, MEN10755 and 4-demethoxy-3-deamino-3-aziridinyl-4-methylsulfonyldaunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzenesulfonamide, anhydrovinblastine, N,N-dimethyl-L-valyl-L-valyl-N-methyl-L-valyl-L-prolyl-L-proline-t-butylamide, TDX258 and BMS188797.

Some examples of topoisomerase inhibitors are topotecan, hycaptamine, irinotecan, rubitecan, 6-ethoxypropionyl-3′,4′-O-exobenzylidenechartreusin, 9-methoxy-N,N-dimethyl-5-nitropyrazolo[3,4,5-kl]acridine-2-(6H)-propanamine, 1-amino-9-ethyl-5-fluoro-2,3-dihydro-9-hydroxy-4-methyl-1H,12H-benzo[de]pyrano[3′,4′:b,7]indolizino[1,2b]quinoline-10,13(9H,15H)-dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxyetoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethyl-amino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)-ethyl)acridine-4-carboxamide, 6-[[2-(dimethylamino)ethyl]amino]-3-hydroxy-7H-indeno[2,1-c]quinolin-7-one and dimesna.

“Antiproliferative agents” include antisense RNA and DNA oligonucleotides, such as G3139, ODN698, RVASKRAS, GEM231 and INX3001, and antimetabolites, such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-di-hydrobenzofuryl)sulfonyl]N′-(3,4-dichlorophenyl)urea, N6-[4-deoxy-4-[N2-[2(E),4(E)-tetradecadienoyl]glycylamino]L-glycero-B-L-mannoheptopyranosyl]adenine, aplidine, ecteinascidin, troxacitabine, 4-[2-amino-4-oxo-4,6,7,8-tetrahydro-3H-pyrimidino[5,4-b]-1,4-thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)tetradeca-2,4,6-trien-9-ylacetic acid ester, swainsonine, lometrexol, dexrazoxane, methioninase, 2′-cyano-2′-deoxy-N4-palmitoyl-1-B-D-arabinofuranosylcytosine and 3-aminopyridine-2-carboxaldehyde thiosemicarbazone. “Antiproliferative agents” also include monoclonal antibodies against growth factors other than those already listed under “angiogenesis inhibitors”, such as trastuzumab, and tumour suppressor genes, such as p53, which can be delivered via recombinant virus-mediated gene transfer (see U.S. Pat. No. 6,069,134, for example). Medicaments according to the invention can also be administered in combination with all other therapeutic antibodies known to the person skilled in the art or pharmaceutical active ingredients which are suitable in connection with the above-mentioned diseases.

Furthermore, anti-EGFR antibody formulations according to the invention can be used for the isolation and for the investigation of the activity or expression of EGFR. In addition, they are particularly suitable for use in diagnostic methods for diseases in connection with unregulated or disturbed EGFR activity.

For diagnostic purposes, antibodies according to the invention can, for example, be radioactively labeled. A preferred labeling method is the iodogen method (Fraker et al., 1978). For diagnostic purposes, the antibody is particularly preferably used as the F(ab′)2 fragment. Excellent results are achieved thereby, meaning that background subtraction is unnecessary. Fragments of this type can be prepared by known methods (e.g., Herlyn et al., 1983). In general, pepsin digestion is carried out in at an acidic pH, and the fragments are separated from undigested IgG and fragments of heavy chains by protein A Sepharose™ chromatography.

The anti-EGFR antibodies in formulations according to the invention preferably exhibit an advantageous biological activity which can easily be determined in enzyme assays, as described in the examples. In enzyme-based assays of this type, the antibodies according to the invention preferably exhibit and cause an inhibiting effect, which is usually documented by IC₅₀ values in a suitable range, preferably in the micromolar range and more preferably in the nanomolar range.

The determination of protein size, structural integrity, purity or glycosylation pattern of the of the antibodies according to the invention according to the invention in formulations according to the invention encompasses, without being restricted thereto, SE-HPLC, peptide mapping (digestion), N-terminal sequencing, SDS-Page, Tris/glycine gradient gel (non-reducing), the FTIR (Fourier transform infrared spectra) method, CD (circular dichroism), RAMAN spectroscopy, carbohydrate staining (PAS method), oligosaccharide profiling, determination of the monosaccharide composition or isoelectric focusing.

The stability of formulations according to the invention can, for example, be determined, without being restricted thereto, with the aid of stability programmes, for example storage at 25° C. and 60% relative atmospheric humidity and at 40° C. and 70% relative atmospheric humidity over an extended period and determination of the stability or structural integrity of the protein at regular intervals, for example by the above-mentioned determination methods (SE-HPLC, FT-IR; SDS-PAGE (reducing or non-reducing)).

Methods for the determination of the biological activity or efficacy of antibodies according to the invention in formulations according to the invention encompass, for example, without being restricted thereto, ELISA, biological cell assays, FTIR or CD.

Methods for the determination of reduced aggregation tendency of highly concentrated formulations according to the invention encompass, for example, without being restricted thereto, visual inspection, sub-visible particles analysis, nephelometry or turbidimetry, dynamic light scattering characterisation.

### EXAMPLE 1

### Preparation of a Highly Concentrated Liquid Anti-EGFR Antibody Formulation by Tangential Flow Filtration (TFF)

380 ml of protein (17 mg/ml in 10 mM phosphate+145 mM NaCl, pH 7.2) are concentrated for 226 min at an entry pressure of 20 psi and an exit pressure of 10 psi by means of a Labscale TFF system (Millipore) with built-in polyether sulfone ultrafiltration membrane having a cut-off of 30 kDa. The retentate obtained has a protein concentration of about 132 mg/ml. The yield is 85%.

or

470 ml of protein (1 7 mg/ml in 10 mM citrate) are concentrated for 226 min at an entry pressure of 20 psi and an exit pressure of 10 psi by means of a Labscale TFF system (Millipore) with built-in polyether sulfone ultrafiltration membrane having a cut-off of 30 kDa. The retentate obtained has a protein concentration of about 123 mg/ml. The yield is 95%.

### EXAMPLE 2

### Preparation of a Highly Concentrated Liquid Anti-EGFR Antibody Formulation by Stirred Ultrafiltration

25 ml of protein (10 mg/ml in 10 mM phosphate+145 mM NaCl, pH 7.2) are concentrated for 144 min at a nitrogen gas pressure of 4 bar by means of an Amicon stirred cell with built-in polyether sulfone ultrafiltration membrane having a cut-off of 30 kDa. The retentate obtained has a protein concentration of about 92 mg/ml. The yield is 95%.

or

25 ml of protein (10 mg/ml in 10 mM citrate, pH 5.5) are concentrated for 168 min at a nitrogen gas pressure of 4 bar by means of an Amicon stirred cell with built-in polyether sulfone ultrafiltration membrane having a cut-off of 30 kDa. The retentate obtained has a protein concentration of about 82 mg/ml. The yield is 95%.

### EXAMPLE 3

### Preparation of a Highly Concentrated Liquid Anti-EGFR Antibody Formulation by Ultrafiltration Under the Action of Centrifugal Forces

15 ml of protein (2 mg/ml in 10 mM phosphate+145 mM NaCl, pH 7.2) are centrifuged at: 2000*g for 90 min in an Ultrafree centrifuge tube (Millipore) with a polyether sulfone ultrafiltration membrane having a cut-off of 30 kDa. The retentate obtained has a protein concentration of about 116 mg/ml. The yield is 95%.

### EXAMPLE 4

### Investigation of Soluble Aggregates of the Highly Concentrated Liquid Anti-EGFR Antibody Formulation

The retentates obtained in Examples 1 to 3 were investigated for the content of soluble aggregates by means of SE-HPLC. The proportion of monomer here after concentration was >99%.

### EXAMPLE 5

### Investigation of Nativity of the Highly Concentrated Liquid Anti-EGFR Antibody Formulation

The retentates obtained in Example 1 were investigated by FT-IR spectrometry. The amide I-2. derivation spectra of the starting material before concentration by tangential flow filtration and of the retentate obtained were congruent here.

1. Process for the preparation of a highly concentrated, liquid formulation comprising at least one anti-EGFR antibody and/or one of its variants and/or fragments by ultrafiltration.

2. Process according to claim 1, characterised in that the highly concentrated, liquid formulation obtained has a content of an anti-EGFR antibody of 10-250 mg/ml.

3. Process according to claim 1, characterised in that the highly concentrated, liquid formulation obtained has a content of an anti-EGFR antibody of 50-180 mg/ml.

4. Process according to claim 1, characterised in that the highly concentrated, liquid formulation obtained has a content of an anti-EGFR antibody of 100-150 mg/ml.

5. Process according to claim 1, characterised in that the anti-EGFR antibody is monoclonal and is of murine or human origin.

6. Process according to claim 1, characterised in that the anti-EGFR antibody is of murine origin and is chimeric or humanised.

7. Process according to claim 1, characterised in that the anti-EGFR antibody is Mab C225 (cetuximab) or Mab h425 (EMD72000).

8. A highly concentrated, liquid formulation comprising at least one anti-EGFR antibody and/or one of its variants and/or fragments.

9. The highly concentrated, liquid formulation according to claim 8, which comprises a content of an anti-EGFR antibody of 10-250 mg/ml.

10. The highly concentrated, liquid formulation according to claim 8, which comprises a content of an anti-EGFR antibody of 50-180 mg/ml.

11. The highly concentrated, liquid formulation according to claim 8, which comprises a content of an anti-EGFR antibody of 100-150 mg/ml.

12. The highly concentrated, liquid formulation according to claim 8, wherein the anti-EGFR antibody is monoclonal and is of murine or human origin.

13. The highly concentrated, liquid formulation according to claim 8, wherein the anti-EGFR antibody is of murine origin and is chimeric or humanised.

14. The highly concentrated, liquid formulation according to claim 8, wherein the anti-EGFR antibody is Mab C225 (cetuximab) or Mab h425 (EMD72000).

15. A highly concentrated, liquid formulation comprising at least one anti-EGFR antibody and/or one of its variants and/or fragments obtainable by a process according to claim 1.

16. A storage stable medicament comprising the highly concentrated, liquid formulation according to claim 8 and a carrier.

17. A composition comprising the highly concentrated, liquid formulation according to claim 8 and an excipient or an adjuvant and optionally a further pharmaceutical active ingredient.

18. (canceled)

19. A method for the treatment of a tumor or a tumor metastasis in a subject in need thereof, comprising administering the highly concentrated, liquid formulation according to claim 8.

20. The method according to claim 19, wherein the tumor is brain tumor, tumour of the urogenital tract, tumor of the lymphatic system, stomach tumor, laryngeal tumor, monocytic leukaemia, lung adenocarcinoma, small-cell lung carcinoma, pancreatic cancer, glioblastoma or breast carcinoma.

21. A highly concentrated, liquid formulation comprising an antibody which is monoclonal antibody c225 (Mab c225) or monoclonal antibody h425 (Mab h425) at a concentration of between 100 mg/ml to 150 mg/ml, wherein said formulation further comprises
at least one agent for reducing viscosity which is sodium chloride, arginine hydrochloride, sodium thiocyanate, ammonium thiocyanate, ammonium sulfate, ammonium chloride, calcium chlorides, zinc chlorides, or sodium acetate; and
optionally at least one stabilizer which is an amino acid, a sugar, a sugar alcohol, an antioxidant, a preservative, a cyclodextrin, an albumin, a polyhydric alcohol or a salt.
